(19) United States
(12) Patent Application Publication
(10) Pub. No.: US 2007/0010873 A1
(43) Pub. Date: Jan. 11, 2007

(54) Aneurysm graft with markers

(75) Inventor: Eugenio Neri, Siena (IT)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: Vascutek Limited, Inchinnan (GB)

(21) Appl. No.: 11/473,723

(22) Filed: Jun. 23, 2006

(30) Foreign Application Priority Data
Jun. 23, 2005 (GB) 0512786.5

(51) Int. Cl. A61F 2/06

(52) U.S. Cl. 623/1.34

(57) ABSTRACT

There is provided an improved graft for treatment of diseased, typically aneurysmal, tissue of the ascending aorta and aortic arch. The graft described is based on the “Elephant Trunk” graft which requires a two-stage procedure for completion of treatment. The modified graft includes a series of markers to allow visualisation of the distal end of the graft, irrespective of the requirements to trim the distal end of the graft to match the patient's anatomy. The markers are typically radio-opaque allowing visualisation by X-ray.

The present application concerns improvements to a graft used to treat aneurysmal disease.

Aortic aneurysm is characterised by the dilation or ballooning out of part of the wall of the aorta, the artery through which blood flows out of the heart to the body. The majority of aortic aneurysms cause little or no symptoms and small aneurysms can be controlled by a reduction in blood pressure achieved through the administration of beta blockers. However, larger aneurysms pose significant dangers to the patient and may require surgery to prevent rupture, which is frequently fatal. An aneurysm may be caused due to genetic conditions or other diseases and forms where the wall of the aorta is weakened, often due to the build up of plaque. High blood pressure can also increase the likelihood of aneurysm development. Rupture of an aortic aneurysm gives only a 20% chance of survival so there is significant emphasis on early diagnosis and treatment.

A number of grafts have been developed to treat a large aneurysm and to reduce the likelihood of rupture. For example EP 1325716 describes a three or four part graft which is assembled in vivo. This graft suffers from the need for complex assembly during surgery. WO 2004/002370 describes a one or two part endovascular graft having intermediate stents along its length.

Borst et al., (in Thorac Cardiovasc Surg (1983) 31:37-40) describes a technique commonly termed the “Elephant Trunk” technique to surgically treat aneurysm in the descending thoracic aorta. This technique has been demonstrated to reduce the risk of multiple stage aortic replacement (see Estrera et al., in Ann Thorac Surg (2002 74:S1803-5).

The “Elephant Trunk” graft is implanted into the patient in a first stage procedure in which the graft is used to replace the ascending aorta and aortic arch and a length of the graft is left hanging in the descending aorta. A second stage procedure involves extending the “Elephant Trunk” graft using a second vascular graft which is anastomosed distally to healthy aorta.

Since the technique requires a two stage process, there remains the possibility of aneurysm rupture before the second stage of the repair can be completed and various improvements to the technique have been suggested (see EP 1245202) and US 2004/0044395. This is especially of concern when the diameter of the aneurysm exceeds 5-6 cms since there is an increased tension at the distal anastomosis due to the mismatch between the graft and the aorta. Although various approaches have been attempted to reduce this problem, a modified “Elephant Trunk” graft has proved to be most successful.

This modified graft (termed the “Dumbo” graft) is characterised by a 70 mm sewing disc (for example of gelatine coated woven polyester) located around the main tube of the graft (see Neri et al., Ann Thorac Surg (2004) 78:E17-18). The sewing disc of the Dumbo graft enables repair of complex aortic lesions that involve both the aortic arch and the descending aorta, even in the presence of a size mismatch between the graft and the aorta at the level of the distal anastomosis. The sewing ring of the graft is able to cover the gap between the aorta and the graft, thus reducing tension on the sutures and avoiding the need to trim fragile tissues of the diseased aorta.

An endovascular device such as a stent is suggested for use to perform the second stage of the procedure. When performing the endovascular second stage, the distal aorta is not visible and the procedure relies on X-ray fluoroscopy. The endovascular device (for example a stent) is designed to be visible under X-ray and carries radio-opaque markers. However, it is important to establish the relative positions of the endovascular device and the previously inserted graft. Even if the second stage is performed surgically, it is useful to be able to identify the location of the distal end of the first stage graft (the Dumbo graft) to assist with planning of the second stage.

Neri et al., (supra) suggests marking the distal end of the first graft with metal clips, during the operation, for this purpose. These clips cannot be attached to the graft during manufacture, as the graft is trimmed at the time of implant to adjust its length to match the patient's anatomy. The position of the distal end is, therefore, not known until the time of implant.

We have now found that the problem of marking the distal end of the graft can be overcome by the inclusion of a series of spaced radio-opaque markers on the distal portion of the graft during manufacture.

Accordingly, in one aspect the present invention provides an Elephant Trunk graft having a series of spaced radio-opaque markers on the distal portion thereof.

The term “Elephant Trunk” graft refers to any vascular graft suitable to repair thoracic aortic aneurysm. The graft will normally replace the ascending aorta and arch of the aorta. In more detail the graft will include a 20 to 40 mm (usually 25-35 mm, for example 30 mm) diameter graft, optionally having a side branch attached thereto. The usual diameter of the side branch will be 5-15 mm, typically 8-12 mm, for example 10 mm.

Optionally up to 4 branches can be present (to replace the supra aorta trunks).

In one embodiment the markers are disposed in pairs with one marker of each pair located on the upper (cranial) surface of the graft and the second marker of each pair being located on the lower (caudal) surface of the graft. In this embodiment there are thus two diametrically opposed rows of markers.

The spacing of the markers is selected to allow precise placement of the endovascular device (stent), whilst avoiding confusion caused by too many markers. We have found that a longitudinal spacing of 8-12 mm (for example 10 mm) between each markers is satisfactory.

In one embodiment the Elephant Trunk graft includes a sewing ring collar (ie. is a Dumbo graft).

The material used to form the markers is chosen to be both biocompatible and highly visible under X-ray. Suitable materials include non-toxic metals such as gold, platinum or tantalum. Plastics rendered radio-opaque by the use of fillers such as barium sulphate or tungsten powder are also suitable.

The markers may be attached to the graft by sewing and advantageously the markers are sized and shaped to allow the passage of a sewing needle and thread. Preferred shapes include a ring or “doughnut” as well as button-like markers in the form of small plates with holes. Alternatively, the markers can be attached to the outer surface of the graft using adhesive bonding or welding. Optionally the markers could be formed into the body of the graft by incorporating metallic or other radio opaque yarns into the textile structure of the graft.

In use the surgeon would trim the graft to length by cutting between a pair of markers. The last remaining markers then identify the distal end of the graft and correct positioning of the endovascular device can be determined by checking the amount of overlap between the endovascular device and the graft. This is easily assessed by counting the markers from the distal end and advancing the endovascular device until it is inserted to lie between an appropriate pair of markers.

Thus, the present invention further provides a graft as described above for use in replacing diseased (eg. aneurysmal) tissue of the ascending aorta and aortic root. The graft can be trimmed to the required length immediately prior to implant, and the newly formed distal end of the graft will remain visible under X-ray to allow completion of the second graft procedure.

In a further aspect, the present invention provides a method of treating an aortic aneurysm requiring replacement of the aortic root and/or aortic arch in a patient, said method comprising:
i) inserting a graft as described above to replace aneuristic tissue, wherein the distal end of the graft is trimmed to a length suitable for the patient's anatomy immediately prior to insertion;
ii) visualising the trimmed distal end of the graft by X-ray; and
iii) fixing the distal end of the graft by means of a separate endovascular device.

The surgical incision required to insert the graft into the patient in step i) will usually be closed prior to X-ray visualisation of the trimmed distal end of the graft, and such X-ray visualisation facilitates the correct placement of a second, separate, endovascular device to fix the free distal end of the graft to healthy aortic tissue.

The present application will be now further described with reference to the figures in which:

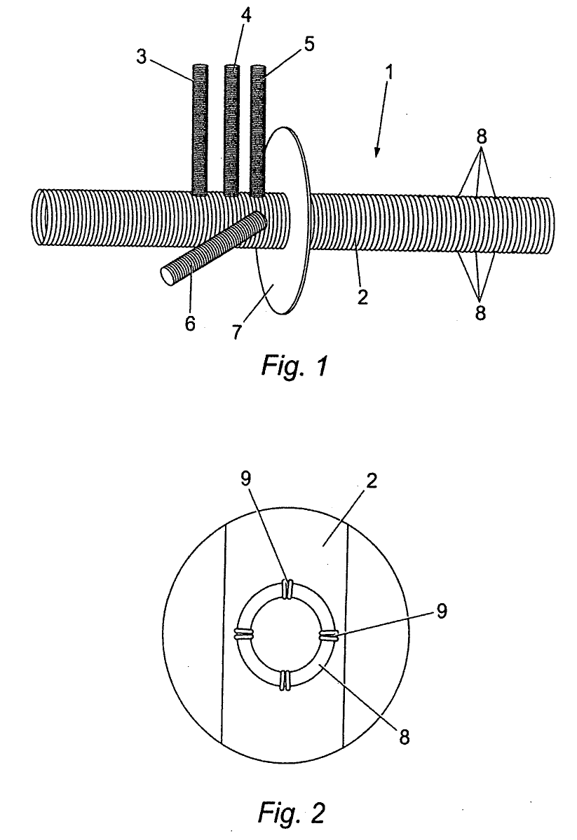

FIG. 1 shows a typical Dumbo graft having radio-opaque markers attached to the distal end thereof; and

FIG. 2 shows an enlarged view of the radio-opaque marker attached to the graft of FIG. 1.

A typical Dumbo graft 1 is shown in FIG. 1 and comprises a main tubular body 2 and three side tubes 3, 4 and 5 which are used to connect to branches of the aorta in the patient. A further side tube 6 is used to allow access to the interior of the graft 1, in particular the lumen of tubular body 2. An endovascular device (stent) is generally introduced down the lumen of the tube 6 during the second stage of the procedure. A sewing ring 7 extends outwardly around the circumference of the tubular body 2 and is located approximately half way along the length of graft 1. Radio-opaque markers 8 are spacedly attached to the distal end of tubular body 2 with a typical spacing of 10 mm between each marker. As illustrated the markers are attached to the outer surface of the graft. However in certain embodiments the markers could be attached to the inner surface of the graft.

As illustrated in FIG. 1, a suitable arrangement has two diametrically opposed rows of markers 8 positioned on body 2. The markers extend along the distal end of the tubular body 2 approximately half way to the sewing ring 7. Suitably each row of markers comprises 4 to 16 markers, preferably 6 to 8 markers.

As shown in more detail in FIG. 2 each of the radio-opaque markers 8 are sewn onto the outer portion of the main body 2 using 4 to 6 equispaced stitches 9. As illustrated, the radio opaque markers are formed from a metal such as tantalum or gold and are in a general doughnut shape. However, other forms of the markers 8 are also possible.

1. An Elephant Trunk graft having a row of spaced radio-opaque markers on the distal portion thereof.

2. The graft as claimed in claim 1 wherein said graft includes a sewing ring for attachment to the aorta.

3. The graft as claimed in claim 1 having two diametrically opposed rows of markers.

4. The graft as claimed in claim 1 having a space of 8 to 12 mm between each marker in a row.

5. The graft as claimed in claim 1 wherein said radio-opaque markers are formed from gold, platinum or tantalum.

6. The graft as claimed in claim 1 wherein said radio-opaque markers are sewn onto the graft.

7. The graft as claimed in claim 1 for use in aortic aneurysm repair.

8. The graft as claimed in claim 1 for use in replacing diseased tissue of the ascending aorta and aortic arch.

9. Method of treating an aortic aneurysm requiring replacement of the aortic root and/or aortic arch in a patient, said method comprising:
i) inserting a graft as claimed in claim 1 to replace aneuristic tissue, wherein the distal end of the graft is trimmed to a length suitable for the patient's anatomy immediately prior to insertion;
ii) visualizing the trimmed distal end of the graft by X-ray; and
iii) fixing the distal end of the graft by means of a separate endovascular device.
